(19) United States
(12) Patent Application Publication
(10) Pub. No.: US 2023/0092731 A1
(43) Pub. Date: Mar. 23, 2023

(54) EMOTION STABILIZATION SUPPORT DEVICE, EMOTION STABILIZATION SUPPORT SYSTEM, AND EMOTION STABILIZATION SUPPORT PROGRAM

(71) Applicant: NIPPON TELEGRAPH AND TELEPHONE CORPORATION, Tokyo (JP)

(72) Inventors: Shingo TSUKADA, Musashino-shi, Tokyo (JP); Kentaro TANAKA, Musashino-shi, Tokyo (JP); Masumi YAMAGUCHI, Musashino-shi, Tokyo (JP); Takayuki OGASAWARA, Musashino-shi, Tokyo (JP); Toichiro GOTO, Musashino-shi, Tokyo (JP)

(73) Assignee: NIPPON TELEGRAPH AND TELEPHONE CORPORATION, Tokyo (JP)

(21) Appl. No.: 17/802,302

(22) PCT Filed: Feb. 28, 2020
(86) PCT No.: PCT/JP2020/008433

§ 371 Date: Aug. 25, 2022

(51) Int. Cl. A61M 21/02; A61B 5/16; A61B 5/11; A63H 33/26

(52) U.S. Cl. CPC A61M 21/02; A61B 5/165; A61B 5/11; A63H 33/26; A61M 2021/0022

(57) ABSTRACT

In an emotion stabilization support device including a portable vibration generator including an oscillator (36), a motion sensor (37), and a control unit (31), the control unit (31) causes the oscillator (36) to intermittently vibrate, and the control unit (31) measures a state of the portable vibration generator detected by the motion sensor (37) and determines a state of an emotion of a user.

### TECHNICAL FIELD

The present invention relates to an emotion stabilization support device, an emotion stabilization support system, and an emotion stabilization support program.

### BACKGROUND ART

There has been proposed an invention for stabilizing an emotion of an infant who becomes emotionally unstable and begins to cry or frets and stopping the infant from crying or calming down the infant.

Patent Literature 1 describes an automatic cradle that detects a cry or the like of an infant and automatically swings. In this automatic cradle, a cradle is swingably pivotally suspended on a base. The cradle is swung using a motor as power. The motor operates according to detection of a cry of an infant by a sound sensor provided in the automatic cradle and swings the cradle.

### CITATION LIST

### Patent Literature

Patent Literature 1: Japanese Patent Laid-Open No. 8-164040

### SUMMARY OF THE INVENTION

### Technical Problem

However, it is not considered that the automatic cradle described in Patent Literature 1 is carried. There is a problem in that it is difficult to use the automatic cradle when going out.

Taking into account the circumstances described above, an object of the present invention is to provide an emotion stabilization support device, an emotion stabilization support system, and an emotion stabilization support program that is portable and supports stabilization of an emotion of an infant.

### Means for Solving the Problem

An emotion stabilization support device according to an aspect of the present invention is an emotion stabilization support device including a portable vibration generator including an oscillator, a motion sensor, and a control unit, wherein the control unit causes the oscillator to intermittently vibrate, and the control unit measures a state of the portable vibration generator detected by the motion sensor and determines a state of an emotion of a user.

An emotion stabilization support system according to an aspect of the present invention includes: a portable vibration generator; a housing instrument that houses the portable vibration generator; and a fixing instrument that fixes the housing instrument to clothes of a user, wherein the portable vibration generator includes an oscillator, a motion sensor, and a control unit, and the control unit is capable of causing the oscillator to intermittently vibrate, measuring acceleration and a posture of the portable vibration generator detected by the motion sensor, and determining a state of an emotion of the user.

An emotion stabilization support program according to an aspect of the present invention is a program for controlling a smartphone including an oscillator, a motion sensor, and a storage unit, the program: causing the oscillator to vibrate in a plurality of vibration patterns; causing the motion sensor to detect acceleration and a posture of the smartphone; causing the storage unit to store the vibration pattern and information concerning the acceleration and the posture of the smartphone at each time; specifying a relation between the vibration pattern and the acceleration and change in the posture of the smartphone; and specifying the vibration pattern for suppressing the acceleration and the change in the posture of the smartphone with respect to the acceleration and the posture of the smartphone.

### Effects of the Invention

With the emotion stabilization support system and the emotion stabilization support program, it is possible to provide an emotion stabilization support device, an emotion stabilization support system, and an emotion stabilization support program that are portable and support stabilization of an emotion of an infant.

### BRIEF DESCRIPTION OF DRAWINGS

FIG. 1 is a perspective view of an emotion stabilization support system according to a first embodiment of the present invention.

FIG. 2 is a functional block diagram of a smartphone of the emotion stabilization support system according to the first embodiment of the present invention.

FIG. 3 is a diagram showing an example of a vibration pattern of the smartphone of the emotion stabilization support system according to the first embodiment of the present invention.

FIG. 4 is a diagram showing an example of a vibration pattern of the smartphone of the emotion stabilization support system according to the first embodiment of the present invention.

FIG. 5 is a diagram showing an example of a vibration pattern of the smartphone of the emotion stabilization support system according to the first embodiment of the present invention.

FIG. 6 is a diagram showing an example of a vibration pattern of the smartphone of the emotion stabilization support system according to the first embodiment of the present invention.

FIG. 7 is a diagram showing an interface screen of the emotion stabilization support system according to the first embodiment of the present invention.

FIG. 8 is a flowchart of a program for controlling the emotion stabilization support system according to the first embodiment of the present invention.

FIG. 9 is a flowchart of a program for controlling the emotion stabilization support system according to the first embodiment of the present invention.

FIG. 10 is a functional block diagram of an emotion stabilization support system according to a second embodiment of the present invention.

FIG. 11 is a functional block diagram of an emotion stabilization support system according to a third embodiment of the present invention.

### DESCRIPTION OF EMBODIMENTS

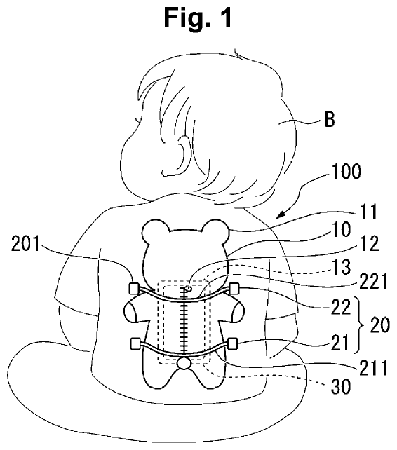

A first embodiment of the present invention is explained with reference to FIG. 1 to FIG. 9.

An emotion stabilization support system 100 according to this embodiment is a system that is portable and gives a vibration stimulus to a user B, who is an infant, to thereby support stabilization of an emotion of the user B.

FIG. 1 is a perspective view of the emotion stabilization support system 100 according to the first embodiment of the present invention. As shown in FIG. 1, the emotion stabilization support system 100 includes a housing instrument 10, a fixing instrument 20, and a smartphone (a portable vibration generator) 30.

The housing instrument 10 is capable of housing the smartphone 30. The housing instrument 10 includes an exterior unit 11, a fastener 12, and a cushion 13. The exterior unit 11 is formed in a bag shape and is capable of housing the smartphone 30 on the inside. The exterior unit 11 has, for example, an external appearance simulating a bear. The exterior unit 11 is formed of, for example, cotton, felt, or a fabric or a knit obtained by using raised cloth.

The fastener 12 makes it possible to open and close the exterior unit 11, house the smartphone 30 in the exterior unit 11, and take out the smartphone 30 housed in the exterior unit 11. The cushion 13 is joined to the inner surface of the exterior unit 11. The cushion 13 is disposed on the opposite side of the fastener 12 in the exterior unit 11. For the cushion 13, an elastic material such as sponge or urethan foam is used.

The fixing instrument 20 fixes the housing instrument 10, for example, substantially in a position between the left and right shoulder bones of the user B. The housing instrument 10 is fixed such that the head of the bear shape is disposed on the head side of the user B and the legs of the bear shape are disposed on the waist side of the user B. The housing instrument 10 is fixed such that the opposite side of the fastener 12 is opposed to the user B.

The fixing instrument 20 includes a lower fixing instrument 21 and an upper fixing instrument 22. The lower fixing instrument 21 fixes the housing instrument 10 such that the leg side of the bear shape of the housing instrument 10 is always in contact with the user B. The lower fixing instrument 21 includes a fixing cord 211 and clips 201. The clips 201 are respectively provided at both ends of the fixing cord 211. The fixing cord 211 is drawn around to the back of the bear shape of the housing instrument 10 and the clips 201 hold the clothes of the user B on the side of the bear shape, whereby the lower fixing instrument 21 fixes the leg side of the housing instrument 10.

The upper fixing instrument 22 fixes the head side of the bear shape of the housing instrument 10. The upper fixing instrument 22 includes a rubber cord 221 and clips 201. The clips 201 are respectively provided at both ends of the rubber cord 221. The rubber cord 221 is drawn around to the back of the bear shape of the housing instrument 10 and the clips 201 hold the clothes of the user B on the sides of the bear shape, whereby the upper fixing instrument 22 fixes the head side of the housing instrument 10.

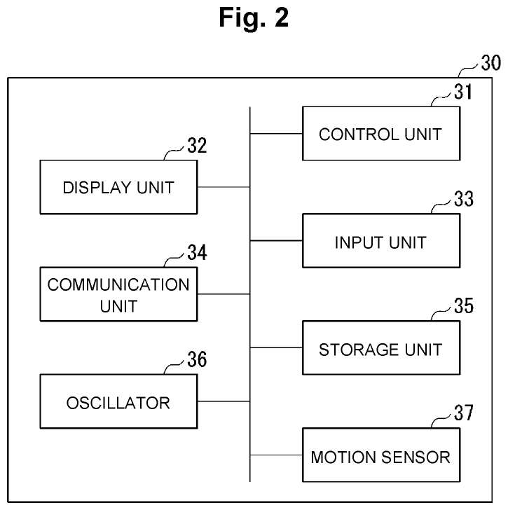

FIG. 2 is a functional block diagram of the smartphone 30. As shown in FIG. 2, the smartphone 30 includes a control unit 31, a display unit 32, an input unit 33, a communication unit 34, a storage unit 35, a vibrator (oscillator) 36, and a motion sensor 37.

The smartphone 30 is a program executable processing device including a not-shown processor, a memory capable of reading a program, a storage device capable of storing a program and data, and an input/output device and includes other dedicated hardware.

The control unit 31 realizes a function of the smartphone 30 by causing the processor to execute a provided program. An emotion stabilization support program explained below is provided to the smartphone 30.

The display unit 32 is, for example, a liquid crystal display or an organic EL (Electro Luminescence) display. The display unit 32 is capable of displaying various kinds of information according to instructions from the control unit 31.

The input unit 33 is, for example, a touch panel and is provided integrally with the display unit 32. The input unit 33 is used to perform a manual input in the program provided to the control unit 31.

For the communication unit 34, a standard such as Bluetooth (registered trademark) or Wi-fi (registered trademark) is used. The communication unit 34 communicates with other equipment. The communication unit 34 is capable of acquiring information from the other equipment and transmitting information to the other equipment according to instructions from the control unit 31.

The storage unit 35 stores various kinds of information. The storage unit 35 stores a program to be provided to the control unit 31 and information and the like acquired from the input unit 33, the communication unit 34, or the motion sensor 37. The storage unit 35 performs storage of information, provision of the stored information to the control unit 31, and the like according to instructions from the control unit 31.

The vibrator 36 vibrates according to, for example, an eccentric rotation mass scheme or a linear resonant actuator scheme. The vibrator 36 vibrates at strength and an interval based on instructions from the control unit 31.

The motion sensor 37 detects acceleration generated in the smartphone 30 and a posture of the smartphone 30. The acceleration and the posture detected by the motion sensor 37 are transmitted to the control unit 31. The control unit 31 detects a movement of the user B carrying the smartphone 30.

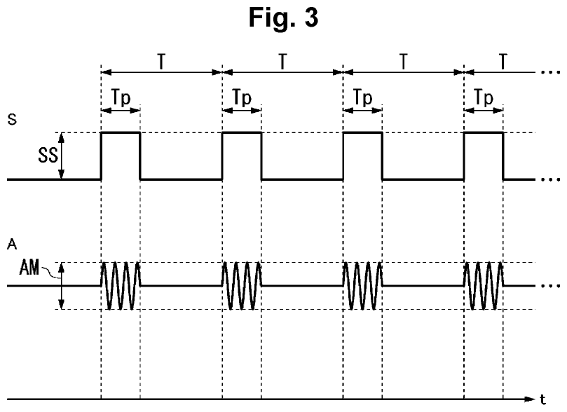

Subsequently, vibration patterns of the vibrator 36 are explained. FIG. 3 to FIG. 6 are examples of the vibration patterns of the vibrator 36. In FIG. 3 to FIG. 6, the horizontal axis indicates time t and the vertical axis indicates strength S of a control signal input to the vibrator 36 from the control unit 31 and amplitude A of vibration of the vibrator 36. The vibrator 36 adjusts the duration of one vibration to thereby adjust the strength of vibration felt by the user B. As the duration of one vibration is longer, the strength of the vibration felt by the user B is larger.

All of the vibration patterns shown in FIG. 3 to FIG. 6 are intermittent vibrations. An interval of vibration is set such that the user B intermittently feels the vibration. In the vibration patterns shown in FIG. 3 to FIG. 6, all of control signals are pulse signals and have constant strength SS. In each vibration pattern, the vibrator 36 vibrates at amplitude AW having constant magnitude. A pulse width is set such that the user B is capable of feeling vibration.

The vibration pattern shown in FIG. 3 is vibration having a constant interval and constant strength (equal interval and equal strength vibration VE). In the equal interval and equal strength vibration VE, the control unit 31 inputs a control signal having a constant pulse width Tp to the vibrator 36 at a constant cycle T. In the equal interval and equal strength vibration VE, the vibrator 36 vibrates in constant duration.

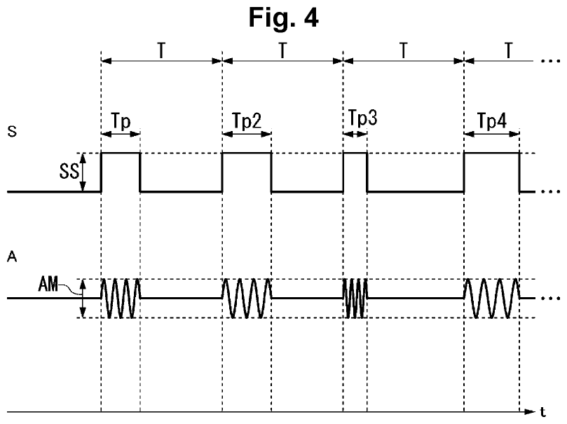

The vibration pattern shown in FIG. 4 is vibration having a constant interval and unequal strength (equal interval and unequal strength vibration VE). In the equal interval and unequal strength vibration VE, the control unit 31 inputs a control signal to the vibrator 36 at the constant cycle T. Pulse widths Tp1, Tp2, Tp3, Tp4, . . . , and Tpn of first to n-th control signals input to the vibrator 36 by the control unit 31 respectively have different magnitudes. In the equal interval and unequal strength vibration VE, the vibrator 36 vibrates at the constant cycle T and in durations different from one another. In the equal interval and unequal strength vibration VE, the control unit 31 repeats the input of the first to n-th control signals to the vibrator 36. A user can set a numerical value of n and pulse widths of the first to n-th control signals.

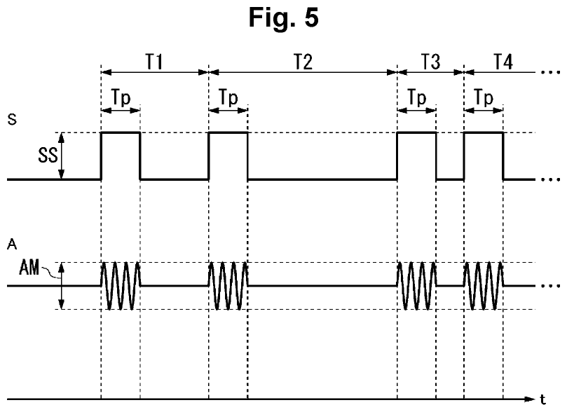

The vibration pattern shown in FIG. 5 is vibration having an unequal interval and having constant strength (unequal interval and equal strength vibration VI). In the unequal interval and equal strength vibration VI, the control unit 31 inputs a control signal having a constant pulse width Tp to the vibrator 36. Intervals T1, T2, T3, T4, . . . , and Tm of control signals input to the vibrator 36 by the control unit 31 respectively have different lengths. In the unequal interval and equal strength vibration VI, the vibrator 36 vibrates at an unequal interval in constant duration. In the equal interval and unequal strength vibration VE, the control unit 31 repeats inputs of first to n-th (n=m+1) control signals to the vibrator 36. The user can set a numerical value of n and intervals of the first to n-th control signals.

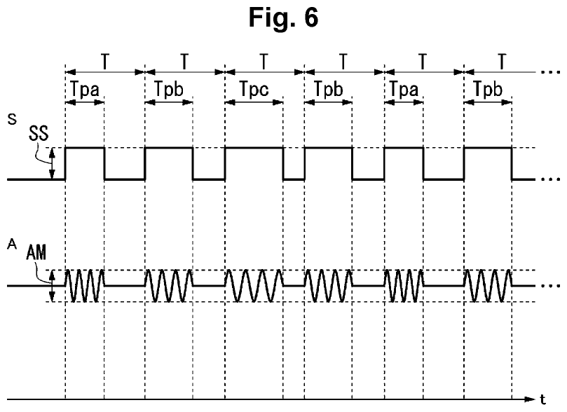

The vibration pattern shown in FIG. 6 is vibration having a constant interval and temporally wavy strength (equal interval and wavy vibration VW). In the equal interval and wavy vibration VW, the control unit 31 inputs a control signal to the vibrator 36 at the constant cycle T. A magnitude relation of pulse widths Tpa, Tpb, and Tpc of control signals input to the vibrator 36 by the control unit 31 is Tpa<Tpb<Tpc. The pulse widths of the control signals input to the vibrator 36 by the control unit 31 are Tpa, Tpb, Tpc, and Tpb in order from the first time to the fourth time. The control unit 31 repeats inputs of the first to fourth control signals to the vibrator 36. Regarding the pulse width of the control signal input to the vibrator 36 by the control unit 31, a cycle in which the pulse width of the control signal gradually increases from a minimum value to reach a maximum value and a cycle in which the pulse width of the control signal gradually decreases from the maximum value to reach the minimum value are repeated.

In the equal interval and wavy vibration VW, in the duration of the vibration of the vibrator 36, a cycle in which the duration gradually increases from the minimum value to reach the maximum value and a cycle in which the duration gradually decreases from the maximum value to reach the minimum value are repeated.

The user can set the minimum values, the maximum values, and the numbers of stages from the minimum values to the maximum values of the control signal and the duration of the vibration.

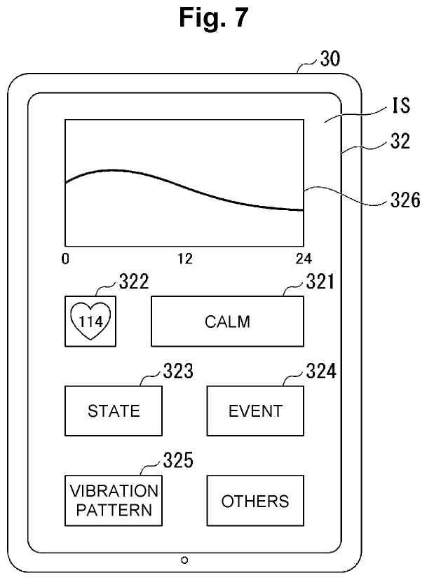

FIG. 7 is a diagram showing an example of an interface screen IS displayed on the display unit 32 of the smartphone 30. As shown in FIG. 7, the interface screen IS includes an emotion determination result display window 321, a heart rate display window 322, an emotion state input button 323, an event input button 324, a vibration pattern input button 325, and a graph 326.

The emotion determination result display window 321 displays an emotion of the user B determined by the control unit 31 of the smartphone 30. In the emotion determination result display window 321, words such as “calm” and “bad mood” or signs or the like representing those emotions are displayed. The control unit 31 determines, from information concerning the acceleration and change in the posture of the smartphone 30 detected by the motion sensor 37, an emotion of the user B based on a program provided to the control unit 31. The acceleration and the change in the posture of the smartphone 30 are caused by a movement of the user B. In general, the range and the speed of the movement of the user B are larger as the emotion of the user B is more unstable. The range and the speed of the movement of the user B are smaller as the emotion of the user B is more stable. The control unit 31 may determine the emotion of the user B from, for example, the acceleration and the change in the posture of the smartphone 30 using a machine learning algorithm such as a random forest.

The heart rate display window 322 is capable of displaying, when the smartphone 30 acquires, via the communication unit 34, information concerning a heart rate detected by attaching a transmitter or the like for measuring a heart rate to the user B, the information of the heart rate.

The emotion state input button 323 is capable of inputting an emotion state of the user B determined by a guardian M or the like of the user B to the smartphone 30. For example, when the guardian M touches the emotion state input button 323, a small window opens and the words such as “calm” and “bad mood” or the signs or the like representing those emotions are displayed. The guardian M touches the words or the signs or the like to input emotion states corresponding thereto.

The event input button 324 is capable of inputting timing when the user B took meal, excreted, or the like.

The vibration pattern input button 325 can select ON/OFF of vibration of the vibrator 36. The vibration pattern input button 325 can select any one vibration pattern among the equal interval and equal strength vibration VE, the equal interval and unequal strength vibration VE, the unequal interval and equal strength vibration VI, or the equal interval and wavy vibration VW explained above. For example, when the guardian M touches the vibration pattern input button 325, a small window opens and ON, OFF, and words of names of the vibration patterns, or signs or the like representing the names are displayed. When the guardian M touches the words or the signs or the like, emotion states corresponding thereto are input.

The vibration pattern input button 325 may be configured such that the guardian M or the like can create a vibration pattern having an interval and strength of any vibration. The vibration pattern input button 325 may be configured such that, for example, a vibration pattern having a high numerical value of evaluation point data Pn explained below can be automatically selected.

The graph 326 displays, as a graph and a sign, information of the display window, input information, and the like.

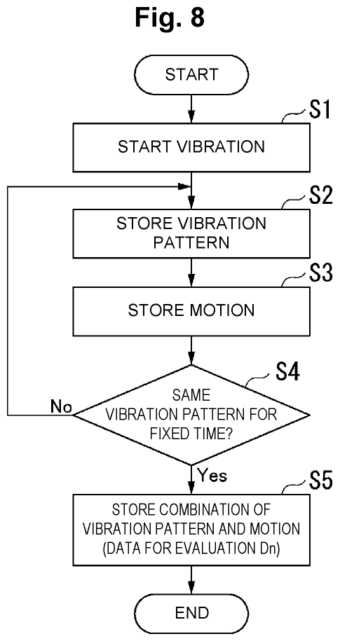

Subsequently, an emotion stabilization support program by vibration of the vibrator 36 is explained. The emotion stabilization support program includes a motion change storage module and an effect evaluation module. FIG. 8 is a flowchart showing a processing procedure of the motion change storage module in an emotion stabilization effect measurement program.

In step S1, the control unit 31 causes the vibrator 36 to vibrate in any one vibration pattern among the equal interval and equal strength vibration VE, the equal interval and unequal strength vibration VE, the unequal interval and equal strength vibration VI, or the equal interval and wavy vibration VW explained above. After performing the processing in step S1, the control unit 31 performs processing in step S2.

In step S2, the control unit 31 causes the storage unit 35 to store in which vibration pattern the control unit 31 causes the vibrator 36 to vibrate. After performing the processing in step S2, the control unit 31 performs processing in step S3.

In step S3, the control unit 31 causes the storage unit 35 to store, for each time, information concerning the acceleration and change in the posture of the smartphone 30 detected by the motion sensor 37. After performing the processing in step S3, the control unit 31 performs processing in step S4.

In step S4, the control unit 31 determines whether a vibration pattern is changed while counting a fixed time. If the vibration pattern is the same and is not changed for the fixed time, the control unit 31 performs processing in step S5. If the vibration pattern is changed in the fixed time, the control unit 31 returns to the processing in step S2.

In step S5, the control unit 31 causes the storage unit 35 to store, as data for evaluation Dn, information obtained by combining information concerning which vibration pattern is stored by the storage unit 35 and the information concerning the acceleration and the change in the posture for each time of the smartphone 30. After performing the processing in step S5, the control unit 31 ends the processing.

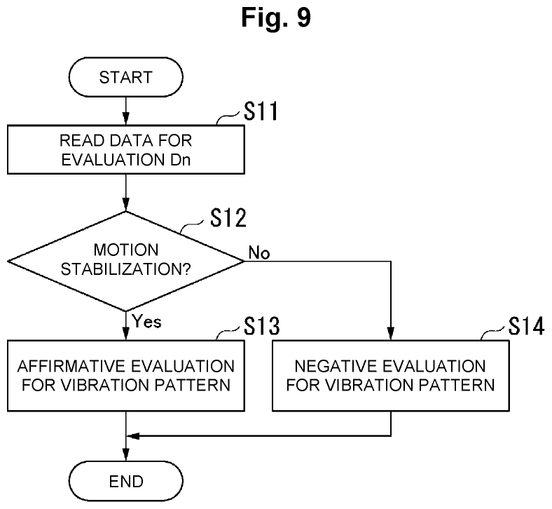

FIG. 9 is a flowchart showing a processing procedure of the effect evaluation module in the emotion stabilization effect measurement program.

In step S11, the control unit 31 reads one data for evaluation Dn. After performing the processing in step S11, the control unit 31 performs processing in step S12.

In step S12, the control unit 31 determines whether the acceleration and the change in the posture of the smartphone 30 decrease according to elapse of time in the read data for evaluation Dn. If the acceleration and the change in the posture of the smartphone 30 decrease according to elapse of time, it is determined that the emotion of the user B is changing from unstable to stable. When determining that the acceleration and the change in the posture of the smartphone 30 decrease according to elapse of time, the control unit 31 performs processing in step S13. When determining that the acceleration and the change in the posture of the smartphone 30 do not decrease according to elapse of time, the control unit 31 performs processing in step S14.

In step S13, the control unit 31 performs an affirmative evaluation for a vibration pattern in the read data for evaluation Dn. For example, the control unit 31 increases a numerical value of evaluation point data Pn stored in the storage unit 35 to be linked with the vibration pattern in the read data for evaluation Dn. After performing the processing in step S13, the control unit 31 ends the processing.

In step S14, the control unit 31 performs a negative evaluation for the vibration pattern in the read data for evaluation Dn. For example, the control unit 31 reduces the numerical value of the evaluation point data Pn stored in the storage unit 35 to be linked with the vibration pattern in the read data for evaluation Dn. After performing the processing in step S14, the control unit 31 ends the processing.

With the emotion stabilization support system 100, it is possible to give a vibration stimulus to the user B, who is an infant, and support stabilization of the emotion of the user B using the portable smartphone 30.

Since the housing instrument 10 housing the smartphone 30 has an external appearance simulating a living thing such as a bear, it is easy to arise an attachment in the user B and stabilize the emotion of the user B.

Since the housing instrument 10 includes the cushion 13, even when the smartphone 30 hits the user B via the housing instrument 10, it is possible to reduce discomfort given to the user B.

The fixing instruments 20 that fixes the housing instrument 10 to the user B includes the lower fixing instrument 21 disposed on the waist side of the user B and the upper fixing instrument 22 fixed to the head side of the user B. The lower fixing instrument 21 fixes the housing instrument 10 to be always in contact with the user B. The upper fixing instrument 22 fixes the housing instrument 10 with the rubber cord 221 having flexibility. Therefore, the housing instrument 10 naturally leans on the user B. The user B has a good wearing feeling.

Since the vibrator 36 is capable of vibrating in the vibration pattern having an unequal interval of vibration or unequal strength of vibration, it is possible to provide, according to the user B, vibration with which the user B easily feels comfortableness.

Since the emotion of the user B can be specified by the motion sensor 37, it is easy to confirm an emotion stabilization effect of the vibration stimulus given to the user B.

Since the emotion stabilization support program is capable of increasing a numerical value of the evaluation point data Pn of a vibration pattern having a high emotion stabilization effect and specifying the vibration pattern having the high emotion stabilization effect, it is easy to stabilize the emotion of the user B.

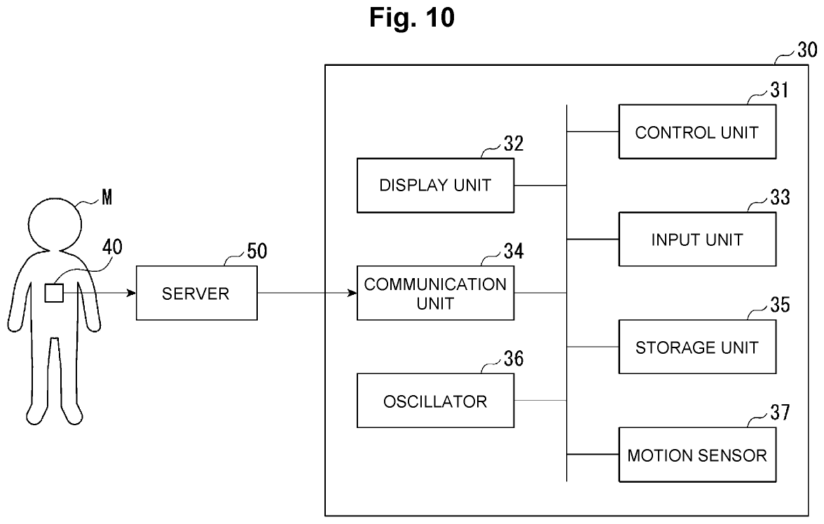

Subsequently, a second embodiment of the present invention is explained with reference to FIG. 10.

An emotion stabilization support system 200 according to this embodiment is a system that supports stabilization of the emotion of the user B by giving, via the portable smartphone 30, a vibration stimulus associated with a heart rate or the like of the guardian M or the like in a remote place to the user B, who is an infant.

FIG. 10 is a functional block diagram of the emotion stabilization support system 200. The emotion stabilization support system 200 includes a biological sensor 40 and a server 50 in addition to the components in the first embodiment. In FIG. 10, description of the housing instrument 10, the fixing instrument 20, and the like is omitted.

The biological sensor 40 is capable of detecting a heart rate, an electrocardiogram, or an interval and strength of respiration or the like of a human body. The biological sensor 40 is attached to, for example, the guardian M present in a remote place from the user B. The biological sensor 40 is capable of communicating with the server 50 and is capable of transmitting information concerning a heart rate, an electrocardiogram, or an interval and strength of respiration or the like of the guardian M detected from the guardian M to the server 50.

The server 50 is capable of communicating with the communication unit 34 of the smartphone 30 and is capable of transmitting the information concerning the heart rate, the electrocardiogram, or the interval and the strength of the respiration or the like of the guardian M acquired from the biological sensor 40 to the communication unit 34.

The smartphone 30 is capable of acquiring the information concerning the heart rate, the electrocardiogram, or the interval and the strength of the respiration or the like of the guardian M from the server 50 via the communication unit 34. The control unit 31 of the smartphone 30 causes the vibrator 36 to vibrate based on the information concerning the heart rate, the electrocardiogram, or the interval and the strength of the respiration or the like of the guardian M.

With the emotion stabilization support system 200, it is possible to support stabilization of the emotion of the user B by giving a vibration stimulus associated with the heart rate or the like of the guardian M or the like in a remote place.

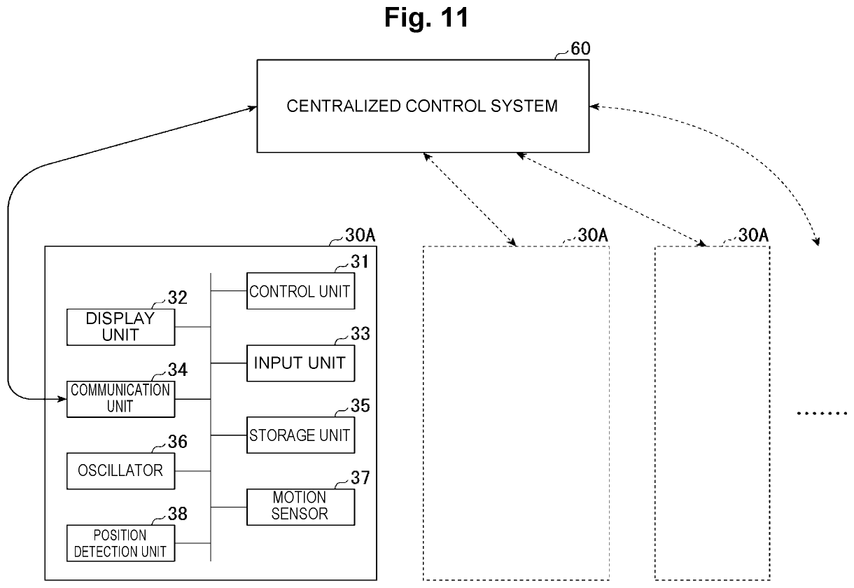

Subsequently, a third embodiment of the present invention is explained with reference to FIG. 11.

An emotion stabilization support system 300 according to this embodiment is a system that supports stabilization of an emotion of the user B by giving a vibration stimulus to the user B, who is an infant, via a portable smartphone 30A.

FIG. 11 is a functional block diagram of the emotion stabilization support system 300. The emotion stabilization support system 300 includes the smartphone 30A instead of the smartphone 30 in the configuration in the first embodiment and further includes a centralized control system 60. In FIG. 11, description of the housing instrument 10, the fixing instrument 20, and the like is omitted.

The smartphone 30A includes a position detection unit 38 in addition to the components of the smartphone 30. The position detection unit 38 specifies a present position of the smartphone 30A with, for example, a GPS (Global Positioning System).

As shown in FIG. 11, the centralized control system 60 is capable of communicating with a plurality of smartphones 30A. The centralized control system 60 communicates with the smartphone 30A via the communication unit 34.

The centralized control system 60 is capable of performing communication with the respective smartphones 30A and acquiring present positions of the respective smartphones 30A. The centralized control system 60 is capable of transmitting, based on the present position of the smartphone 30A, information concerning a vibration pattern set considering characteristics of the present position of the smartphone 30A to the smartphone 30A.

The smartphone 30A acquires the information concerning the vibration pattern matched with the characteristics of the present position and causing the vibrator 36 to vibrate according to the vibration pattern set considering the characteristics of the present position.

With the emotion stabilization support system 300, it is possible to support stabilization of the emotion of the user B by giving a vibration stimulus by the vibration pattern set considering the characteristics of the present position to the user B of the smartphone 30A.

In general, an infant easily falls into a stress state such as an anxiety or a tension and becomes unstable in an emotion at an arrival time at a nursery or a day nursery, a moving time into a train or an airplane or a landing time of the airplane that the infant is boarded, or the like. The infant easily becomes stable in an emotion in the nursery or the day nursery, the train or the airplane in a constant speed operation state, or the like. Therefore, when the present position of the user B is a place where the emotion of the user B easily becomes unstable, it is possible to accurately stabilize the emotion of the user B by the centralized control system 60 providing a vibration stimulus having high strength that easily stabilizes the emotion of the user B. When the present position of the user B is a place where the emotion of the user B easily becomes stable, it is possible to maintain stabilization of the emotion of the user B by the centralized control system 60 providing a vibration having a long interval and low strength.

The embodiments of the present invention are explained in detail above with reference to the drawings. However, a specific configuration is not limited to the embodiments. A design change and the like in a range not departing from the gist of the present invention are also included in the specific configuration. The constituent elements described in the embodiment explained above and modifications explained below can be combined as appropriate and configured.

For example, an emotion stabilization support device may not include the housing instrument 10 and the fixing instrument 20 and may be configured by a portable vibration generator.

The portable vibration generator may not be the smartphone and may not have a telephone function and the like. For example, the portable vibration generator may be configured using portable information equipment such as a portable game machine, a wearable computer, or a dedicated device.

The oscillator may not be the vibrator 36 and may be a component that emits sound as vibration such as a speaker.

The housing instrument may have an external appearance simulating, other than the bear, a living thing such as a human, an animal, or an insect, a product such as a vehicle or food, or the like. The housing instrument may be a bag such as a backpack shouldered by a doll simulating the living thing, the product, or the like. The housing instrument may be a bag such as a purse or a backpack not simulating the living thing, the product, or the like.

The fixing instrument may not include the fixing cord 211 and the rubber cord 221 and may be the clips 201 attached to the housing instrument. The clips 201 attached to the housing instrument may grab clothes or the like of a user and fix the housing instrument to be suspended from the user.

The fixing instrument may not include the fixing cord 211, the rubber cord 221, and the clips 201 and may be a hook-and-loop fastener, a snap button, or the like provided in the clothes of the user B or the clothes of the user B such as a baby sling and the housing instrument. The housing instrument and the fixing instrument may be a pocket formed in the clothes of the user B. The fixing instrument may be a backpack or the like. The fixing instrument only has to be an attachment instrument used when the user wears the housing instrument 10.

A vibration pattern may have vibration having an unequal interval and temporally wavy strength.

The emotion stabilization support system may measure a state other than the acceleration and the posture of the portable vibration generator if emotion stabilization support system can determine a state of the emotion of the user B.

The emotion stabilization support system may be configured such that a biological signal of a heart rate, an electrocardiogram, respiration, a posture, a motion, a state of an emotion, and the like of the user B is acquired by the motion sensor 37 or the biological sensor 40 and a vibration pattern of the vibrator is determined based on the biological signal of the user B.

The emotion stabilization support system may cause the control unit to determine a state of use of the emotion stabilization support system based on information concerning vibration of the portable vibration generator detected by the motion sensor. The control unit may determine that the vibration of the portable vibration generator is weakened because, for example, the portable vibration generator is pressed by the user, the guardian, or the like and determine a state of use of the emotion stabilization support system.

The position detection unit may detect a beacon, an ID of a wireless base station, a change in radio intensity, or the like and specify a present position of the portable vibration generator.

The centralized control system may transmit information concerning a vibration pattern to the portable vibration generator as area information. The centralized control system may transmit information concerning a vibration pattern set according to characteristics of present time to the portable vibration generator.

The centralized control system may acquire the evaluation point data Pn of a plurality of portable vibration generators and transmit a vibration pattern having a high evaluation in a plurality of users B to the plurality of portable vibration generators.

### REFERENCE SIGNS LIST

10 Housing instrument

20 Fixing instrument

30, 30A Smartphone (Portable vibration generator)

31 Control unit

32 Display unit

33 Input unit

34 Communication unit

35 Storage unit

36 Vibrator (Oscillator)

37 Motion sensor

38 Position detection unit

40 Biological sensor

50 Server

60 Centralized control system

100, 200, 300 Emotion stabilization support system

1. An emotion stabilization support device comprising a portable vibration generator including an oscillator, a motion sensor, and a control unit, wherein
the control unit causes the vibrator to intermittently vibrate, and
the control unit measures a state of the portable vibration generator detected by the motion sensor and determines a state of an emotion of a user.

2. An emotion stabilization support system comprising:
a portable vibration generator;
a housing instrument that houses the portable vibration generator; and
an attachment instrument used when the user wears the housing instrument, wherein
the portable vibration generator includes an oscillator, a motion sensor, and a control unit, and
the control unit causes the vibrator to intermittently vibrate, and
the control unit measures a state of the portable vibration generator detected by the motion sensor and determines a state of an emotion of the user.

3. The emotion stabilization support system according to claim 2, wherein the portable vibration generator is portable information equipment.

4. The emotion stabilization support system according to claim 2, wherein the housing instrument has an external appearance simulating a living thing or a product.

5. The emotion stabilization support system according to claim 2, wherein the control unit controls strength and an interval of the vibration.

6. The emotion stabilization support system according to claim 5, wherein
the portable vibration generator includes a communication unit, and
the control unit determines the strength and the interval of the vibration based on information obtained via the communication unit.

7. The emotion stabilization support system according to claim 5, wherein
the portable vibration generator includes a position detection unit, and
the control unit determines the strength and the interval of the vibration based on information concerning a present position of the user obtained via the position detection unit.

8. An emotion stabilization support program for controlling portable information equipment including an oscillator, a motion sensor, and a storage unit,
the program:
causing the vibrator to vibrate in a plurality of vibration patterns;
causing the motion sensor to detect acceleration and a posture of the portable information equipment;
causing the storage unit to store the vibration pattern and information concerning the acceleration and the posture of the portable information equipment at each time;
specifying a relation between the vibration pattern and the acceleration and change in the posture of the portable information equipment; and
specifying the vibration pattern for suppressing the acceleration and the change in the posture of the portable information equipment with respect to the acceleration and the posture of the portable information equipment.
